(19) United States
(12) Patent Application Publication
(10) Pub. No.: US 2026/0162813 A1
(43) Pub. Date: Jun. 11, 2026

(54) BIOMETRIC IRIS SCAN TO AUTHENTICATE USERS IN HEALTHCARE SETTINGS

(71) Applicant: Alcon Inc., Fribourg (CH)

(72) Inventor: Ankur Kirit Kapadia, Sioux Falls, SD (US)

(21) Appl. No.: 19/409,170

(22) Filed: Dec. 4, 2025

Related U.S. Application Data
(60) Provisional application No. 63/728,976, filed on Dec. 6, 2024.

(51) Int. Cl. G16H 40/20; G16H 10/60; G16H 40/63; G16H 70/20

(52) U.S. Cl. CPC G16H 40/20; G16H 10/60; G16H 40/63; G16H 70/20

(57) ABSTRACT

The present disclosure generally relates to a system and method of authenticating a provider prior to performing a procedure. The system includes an optometric or ophthalmic device, an iris imager, and one or more processors. The one or more processors acquire, using the iris imager, an image of an iris of a provider, and attempt to authenticate the provider using a comparison of the image of the iris with a reference iris scan of the provider. Responsive to authenticating the provider, the one or more processors access a set of one or more procedures approved for the provider. Responsive to determining that a procedure is included in the set of one or more procedures, the one or more processors configure operation of the optometric or ophthalmic device to perform the procedure.

### CROSS-REFERENCE TO RELATED APPLICATIONS

This application claims the benefit of U.S. Provisional Application Ser. No. 63/728,976 (filed on December 6, 2024), the content of which is incorporated by reference herein in its entirety.

### BACKGROUND

Within healthcare settings, such as a clinical or surgical environment, the authentication of providers (e.g., surgeons, nurses, anesthesiologists) and patients is important to ensure safety, accuracy, and compliance with laws and regulations. Authentication of patients reduces a likelihood of misidentification, which can cause errors such as wrong-site surgery, incorrect procedures, or incorrect administration of anesthesia or other medications. Authentication of providers ensures that those performing (or supporting) a procedure are properly authorized and credentialed, and also documents those involved in the procedure, which is helpful for post-procedure care and auditing. Further, various laws and regulations (e.g., the Health Insurance Portability and Accountability Act (HIPAA) in the U.S.) require authentication mechanisms to ensure patient confidentiality and to prevent unauthorized access to patient records or surgical environments.

Some examples of authentication techniques include printed wristbands or tags, manual entry of passwords or passcodes, fingerprint scans, radio frequency identification (RFID) enabled cards, wristbands, or tags, multi-factor authentication e.g., using an electronic device of the provider, and so forth. However, these forms of authentication can have significant drawbacks. For example, certain forms require additional touching of surfaces or devices (e.g., keyboards, touchscreens, fingerprint scanners, RFID cards), which can make maintaining a sterile environment challenging. Certain forms can increase the cognitive load on providers (e.g., remembering complex passwords, remembering to carry an RFID card) and/or cause delays or interruptions to the procedure. Further, providers may be inclined to circumvent authentication procedures (e.g., sharing passwords or RFID cards) to mitigate some of these drawbacks.

### SUMMARY

The present disclosure generally relates to techniques for authenticating providers and/or patients to prior to performing procedures using an optometric or ophthalmic device. In some aspects, the method includes acquiring, using an iris imager that is communicatively connected with the optometric or ophthalmic device, an image of an iris of the provider; attempting to authenticate the provider using a comparison of the image of the iris with a reference iris scan of the provider; responsive to authenticating the provider, accessing a set of one or more procedures approved for the provider; and responsive to determining that the procedure is included in the set of one or more procedures, configuring operation of the optometric or ophthalmic device to perform the procedure. In some aspects, the iris imager is: integrated into the optometric or ophthalmic device; attached to the optometric or ophthalmic device; or integrated into a body-worn device and wirelessly connected to the optometric or ophthalmic device.

The techniques enable biometric data to be used to authenticate providers without disruptions and delays to the procedure, reducing cognitive load on providers while minimizing a risk of unauthorized access. Further, once authenticated, the optometric or ophthalmic device is configured to perform only one or more of a set of approved procedure(s) that the providers are associated with, thereby reducing the risks of performing an incorrect procedure or errors within the procedure. Although procedures using an optometric or ophthalmic device are discussed as examples of procedures that may benefit from the described aspects, the advantages of the surgical devices and systems described herein may benefit procedures in other clinical and/or surgical environments as well.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope, and may admit to other equally effective aspects.

FIG. 1 illustrates a block diagram of an example system for performing a procedure using an optometric or ophthalmic device, according to one or more aspects.

FIG. 2A illustrates a perspective view of an example optometric device, according to one or more aspects.

FIG. 2B illustrates a perspective view of another example optometric device, according to one or more aspects.

FIG. 3 illustrates a perspective view of an example ophthalmic system, according to one or more aspects.

FIG. 4A illustrates a perspective view of an example body-worn device, according to one or more aspects.

FIG. 4B illustrates a perspective view of another example body-worn device, according to one or more aspects.

FIG. 5 is a method of authenticating a provider and, optionally, a patient relative to a procedure using an optometric or ophthalmic device, according to one or more aspects.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

The present disclosure generally relates to techniques for authenticating providers and/or patients to perform procedures using an optometric or ophthalmic device.

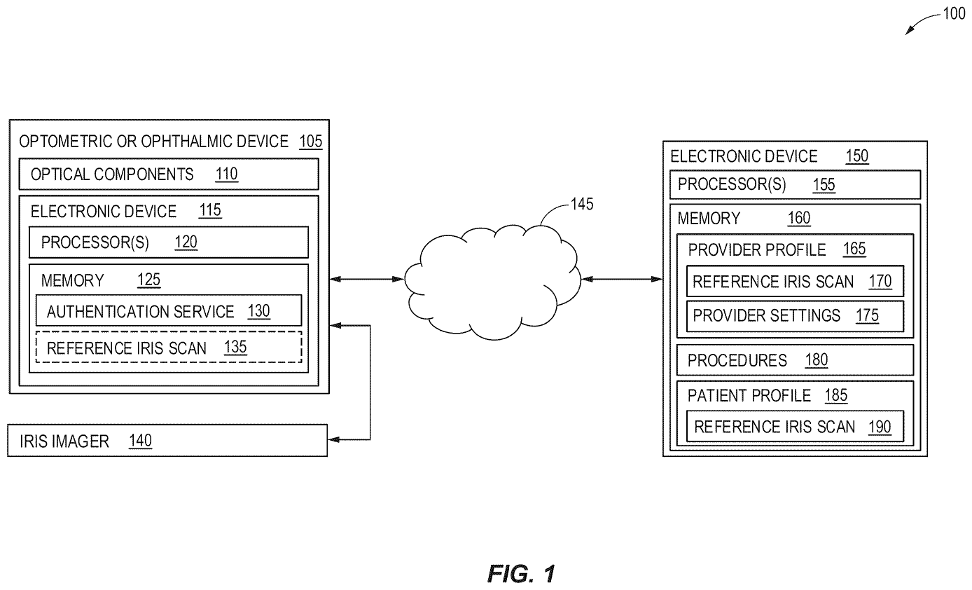

FIG. 1 illustrates a block diagram of a system 100 for performing a procedure using an optometric or ophthalmic device, according to one or more aspects. The system 100 comprises an optometric or ophthalmic device 105 that is communicatively connected with an electronic device 150 via a network 145.

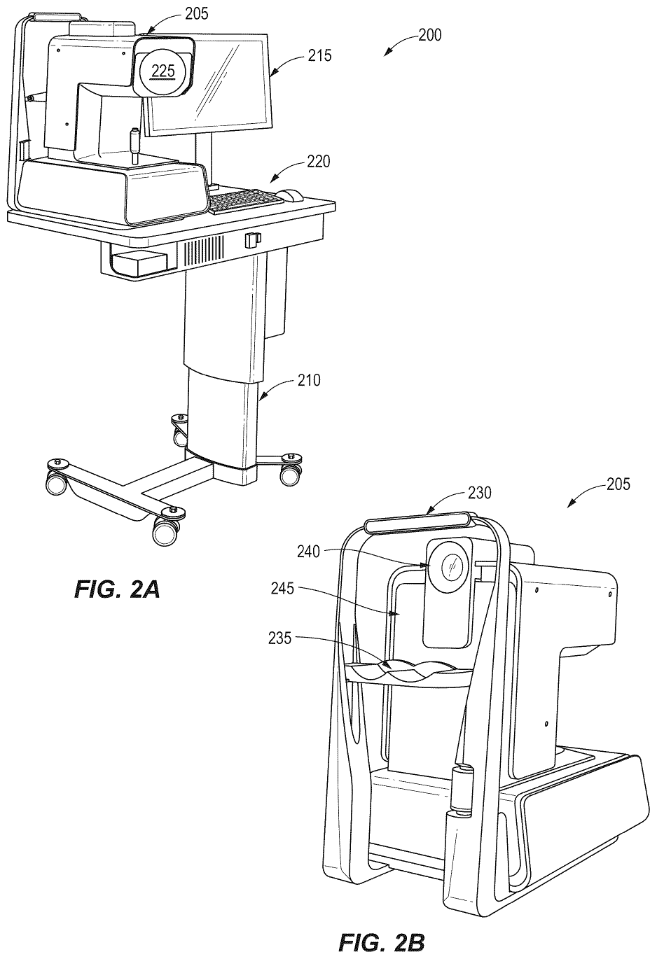

The optometric or ophthalmic device 105 may have any implementation that is suitable for operation by one or more providers within (or in conjunction with) a procedure, such as a testing device, a diagnostic device, a visualization device, a surgical device, and so forth, as well as combinations thereof. Some non-limiting examples of the optometric or ophthalmic device 105 are depicted in FIGS. 2A, 2B, 3, 4A, and 4B.

The optometric or ophthalmic device 105 comprises one or more optical components 110, such as one or more sensors, detectors, light sources, lenses, mirrors, beam splitters, beam combiners, projection systems, reflecting prisms, dispersing devices, filters and thin films, fiber optics, and/or the like, for performing one or more functions of the exemplary optometric or ophthalmic device 105.

In some aspects, the optometric or ophthalmic device 105 comprises an electronic device 115 having at least one processor 120 and a memory 125. The electronic device 115 is communicatively connected with an iris imager 140. In some aspects, the iris imager 140 is integrated into the optometric or ophthalmic device 105; attached to the optometric or ophthalmic device 105; or integrated into a body-worn device and wirelessly connected to the optometric or ophthalmic device 105.

In some aspects, the iris imager 140 comprises one or more cameras that are configured to capture an image of an iris of a provider. In some aspects, the one or more cameras capture images at a distance of about 10-40 centimeters (cm) from the eye, which allows the images to be captured with minimal contact of the provider to devices or surfaces, making the configuration well-suited for seamless use even within a sterile environment.

In some aspects, the iris imager 140 comprises a light source that directs infrared (IR), near-IR light, or visible light into the eye of the provider. The ocular structures within the iris cause light to be reflected onto a sensor of the iris imager 140 (e.g., an infrared sensor, a visible light sensor) to form the image. The iris imager 140 may further include or be configured with hardware and software for performing processing on the captured image to determine whether the scan of the iris has sufficient quality. For example, the processing may assess a sharpness of the image, an iris-sclera contrast, an iris-pupil contrast, pupillary dilation, and/or the presence of any artifacts such as eyelashes or eyelid occlusions. The processing may further detect and/or localize the iris within the image. In other aspects, the hardware and software may be implemented outside of the iris imager 140 (e.g., as part of the authentication service 130).

The memory 125 comprises an authentication service 130 that authenticates a provider for a particular procedure using the image of the iris acquired by the iris imager 140. In some aspects, the authentication service 130 authenticates the provider further using information stored in a memory 160 of the electronic device 150, and accessed via the network 145.

The network 145 may have any suitable implementation, such as one or more wide area networks (WANs), one or more local access networks (LANs), or combinations thereof. The network 145 comprises infrastructure for communicative capability, such as conductive cabling, wireless transmission, optical transmission, and so forth. The network 145 may further comprise one or more electronic devices providing network functionality and/or services to the network 145, such as routers, firewalls, switches, gateway computers, edge servers, and so forth.

As shown, the electronic device 150 comprises at least one processor 155 and a memory 160 that stores one or more provider profiles 165, each including a reference iris scan 170 for the respective provider, and provider settings 175 that reflect one or more preferences for the respective provider (e.g., preferred or historical configuration settings for the optometric or ophthalmic device 105). The electronic device 150 may have any suitable form, and in some embodiments is implemented as a remote server.

In some aspects, the memory 160 further stores information for one or more procedures 180 in any suitable form, such as structured data, semi-structured data, or unstructured data. For example, the one or more procedures 180 may include fields and/or values for a name of the procedure, a provider, a patient, a scheduled date and time, a location, equipment identification information (e.g., corresponding to the optometric or ophthalmic device 105), equipment settings information, and so forth. In some aspects, the memory 160 further stores information for one or more patient profiles 185 that each include a reference iris scan 190 for the respective patient.

The authentication service 130 acquires the image of the iris from the iris imager 140, and compares the image with a reference iris scan 135, 170 of the provider. In some aspects, the reference iris scan 135 is stored locally in the memory 125 of the electronic device 115. In some other aspects, the reference iris scan 135 is a copy of reference iris scan 170 and is retrieved from the electronic device 150 via the network 145.

In some aspects, the image of the iris is compared with a plurality of reference iris scans, and the information associated with a matching reference iris scan is used to ascertain the identity of the provider. In yet some other aspects, an identity of the provider is first ascertained by the authentication service 130 (e.g., information for one or more procedures 180 is retrieved from the electronic device 150, and the identify of a provider for a scheduled procedure is selected) and the image of the iris is compared with the reference iris scan 135, 170 associated with the provider.

In some aspects, the authentication service 130 may be further configured to perform one or more other authentication methods, e.g., when authentication using the image of the iris is unsuccessful. For example, responsive to an unsuccessful authentication, the authentication service 130 may prompt the provider for input at an input device that is communicatively connected with the optometric or ophthalmic device 105 (e.g., a keyboard or PIN pad) or that is communicatively connected to the network 145.

In some aspects, the authentication service 130 may be further configured to authenticate the patient, whether using the iris imager 140 or another device and/or method. In some aspects, the iris imager 140 or another imaging device is operated to acquire an image of an iris of the patient, and the authentication service 130 compares the image of the iris of the patient is compared with the reference iris scan 190 of the patient.

Responsive to authenticating the provider (and in some cases, also authenticating the patient), the authentication service 130 accesses a set of one or more procedures (of the procedures 180) that are approved for the provider. In some aspects, responsive to determining that the procedure to be performed by the optometric or ophthalmic device 105 is included in the set of one or more procedures, the authentication service 130 configures operation of the optometric or ophthalmic device 105 to perform the procedure. In some aspects, configuring operation of the optometric or ophthalmic device 105 to perform the procedure comprises transmitting a control signal that enables operation or controls one or more operational parameters of the optometric or ophthalmic device 105.

FIGS. 2A and 2B illustrate perspective views of an example optometric device 205, according to one or more aspects. The features illustrated in FIGS. 2A and 2B may be used in conjunction with other aspects. For example, the optometric device 205 represents one example of the optometric or ophthalmic device 105 of FIG. 1. Some non-limiting examples of the optometric or ophthalmic device 105 include an optical biometer, an optical coherence tomography (OCT) instrument such as a swept source-OCT (SS-OCT) biometer, an OCT retina and/or anterior segment imaging system, other low-coherence interferometry instrument, a conventional or wide-angle fundus camera for taking images of the eye, a digital fundus camera, an instrument for fundus autofluorescence (AF) and/or multispectral imaging, an instrument for taking measurements of the eye for diagnosis or pre-operative planning such as for cataract or vitreoretinal surgery, an instrument for generating data about the eye, a keratometer, an autorefractor, a topography measurement device such as a corneal topography device, a device for tear film assessment, an ultrasound device such as a B-scan ultrasound device, or combinations thereof. Other types of preoperative, perioperative (i.e., interoperative), and/or postoperative devices are also contemplated.

FIG. 2B depicts a first side of the optometric device 205 that faces the patient during a procedure, allowing the eye(s) of the patient to be suitably positioned for the procedure. As shown in FIG. 2B, the optometric device 205 is an upright optical biometer having a headrest 230 for positioning a forehead of the patient, a chin rest 235 for positioning a chin of the patient, and a measurement module 245. The optometric device 205 further includes an optical system 240 (for example, within the measurement module 245) that includes a succession of optical components (e.g., the optical components 110 of FIG. 1), such as one or more sensors, detectors, light sources, lenses, mirrors, beam splitters, beam combiners, projection systems, reflecting prisms, filters and thin films, fiber optics, and/or the like, for performing one or more diagnostic functions of the optometric device 205.

FIG. 2A depicts a second side of the optometric device 205 that faces the provider during a procedure. In the optometric system 200, the optometric device 205 is disposed on a table 210, along with a display 215 and input devices 220 that are communicatively connected with the optometric device 205. In some aspects, the display 215 displays a graphical user interface (GUI) allowing the provider to view and/or control one or more aspects of the optometric device 205 using the input devices 220. The input devices 220 are shown as a keyboard and mouse; however, other types of input devices are also contemplated, such as a touchscreen integrated with the display 215, a voice-controlled device, and so forth.

An iris imager 225 representing one example of the iris imager 140 is oriented toward the second side of the optometric device 205 to acquire image(s) of an iris of the provider to authenticate the provider prior to, during, and/or after the procedure. In some aspects, the iris imager 225 is integrated into or attached to the optometric device 205. In some other aspects, the iris imager 225 may be separate from the optometric device 205, such as integrated into or attached to the display 215, and communicatively connected to the optometric device 205. Thus, in various configurations, the iris imager 225 may be capable of unobtrusively acquiring images of the iris of the provider while the provider approaches or is positioned at the optometric system 200. In some aspects, the authentication service 130 is further configured to authenticate the patient using optical components included in the optical system 240 or using an iris imager oriented toward the first side of the optometric device 205. In one non-limiting example, the iris imager 225 may be pivoted or otherwise reoriented to acquire images of the irises of the provider and of the patient to authenticate the provider and the patient for performing the procedure.

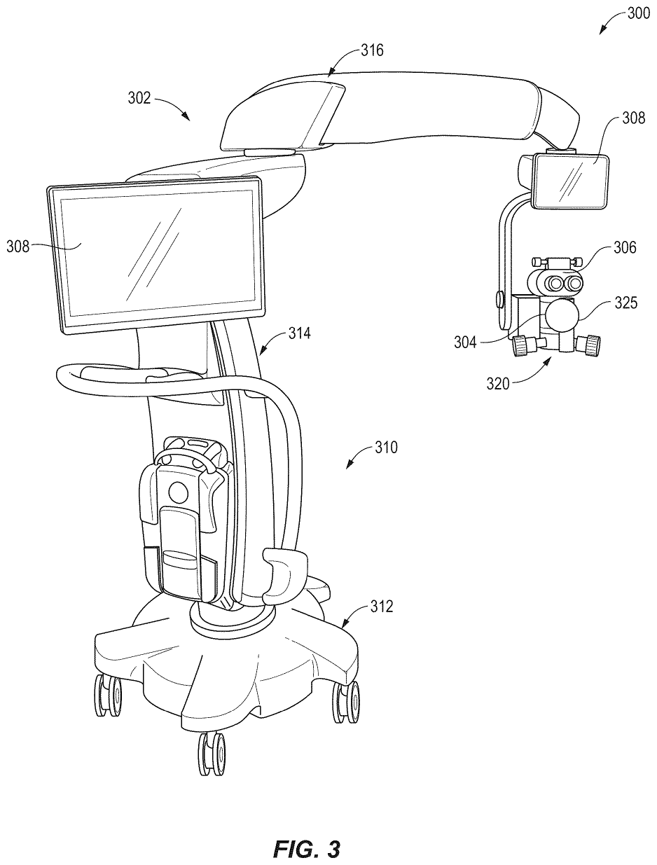

FIG. 3 illustrates a perspective view of an example ophthalmic system 300, according to one or more aspects. The features illustrated in FIG. 3 may be used in conjunction with other aspects. For example, the surgical microscope 302 of the ophthalmic system 300 represents one example of the optometric or ophthalmic device 105 of FIG. 1. Some other non-limiting examples of the optometric or ophthalmic device 105 include a microscope for magnification of the eye and its structures, a camera for taking images of the eye, an OCT system, a fundus autofluorescence system, a slitlamp biomicroscope, an ophthalmoscope, an ultrasound device, a device for visual field testing such as microperimetry, or combinations thereof. Other types of preoperative, perioperative, and/or postoperative devices are also contemplated.

As shown, the surgical microscope 302 comprises a floor stand 310 that includes a rolling base 312, a stanchion 314 projecting upward from the rolling base 312, and an articulating arm 316 projecting laterally from an upper end of the stanchion 314. The surgical microscope 302 further comprises a microscope head 304, oculars 306, and one or more integrated displays 308 that are supported by the articulating arm 316 and/or the stanchion 314; however, the description below is equally applicable to other types of optometric or ophthalmic imaging and/or visualization devices, including those described above. The surgical microscope 302 further comprises an optical system 320 (for example, within the microscope head 304) that includes a succession of optical components (e.g., the optical components 110 of FIG. 1), such as one or more sensors, detectors, light sources, lenses, mirrors, beam splitters, beam combiners, projection systems, reflecting prisms, dispersing devices, filters and thin films, fiber optics, and/or the like, for performing one or more functions of the ophthalmic system 300. In certain embodiments, the one or more displays 308 are configured to display a graphical user interface (GUI) for controlling one or more aspects of the ophthalmic system 300.

An iris imager 325 representing one example of the iris imager 140 is oriented toward a first side of the surgical microscope 302 to acquire image(s) of an iris of the provider to authenticate the provider prior to, during, and/or after the procedure. In some aspects, the iris imager 325 is integrated into or attached to the surgical microscope 302 near the microscope head 304. In other aspects, the iris imager 325 may be integrated into or attached to other component(s) of the surgical microscope 302. Thus, the iris imager 325 may be capable of unobtrusively acquiring images of the iris of the provider while the provider approaches or is positioned at the ophthalmic system 300. In some aspects, the authentication service 130 is further configured to authenticate the patient, using optical components included in the surgical microscope 302 or using an iris imager oriented toward a second side of the surgical microscope 302 that faces the patient during a procedure, e.g., beneath the optical system 320. In one non-limiting example, the iris imager 325 may be pivoted or otherwise reoriented to acquire images of the irises of the provider and of the patient to authenticate the provider and the patient for performing the procedure.

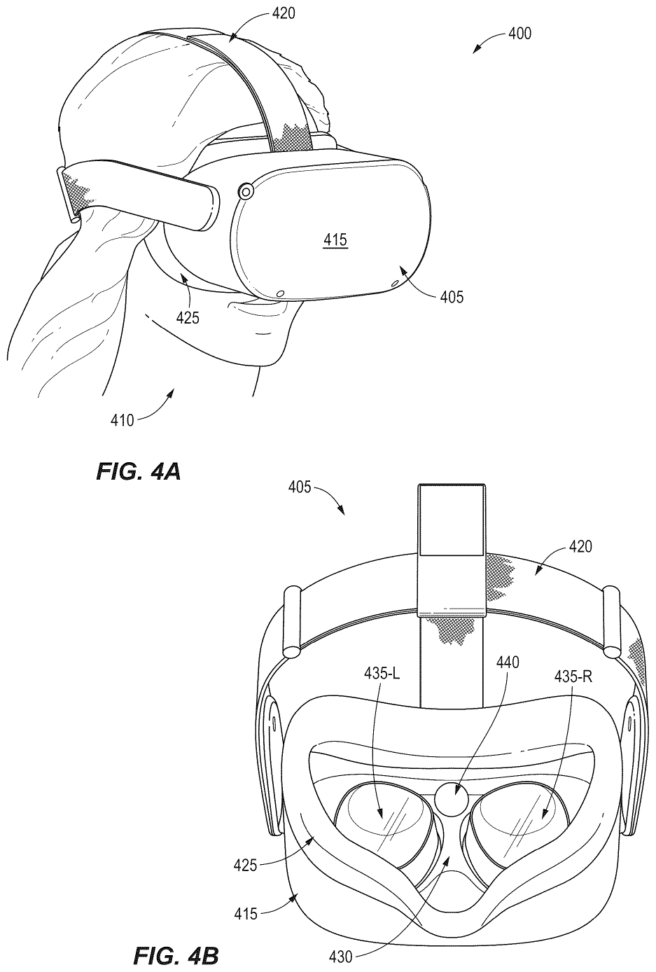

FIGS. 4A and 4B illustrate perspective views of an example body-worn device 405, according to one or more aspects. The features illustrated in FIGS. 4A and 4B may be used in conjunction with other aspects. For example, the body-worn device 405 may be worn by a provider and wirelessly connected with the optometric or ophthalmic device 105.

As shown, the body-worn device 405 is implemented as a virtual reality (VR) headset comprising a body 415, and a strap assembly 420 that retains the body 415 on the head of a wearer 410. The body-worn device 405 further comprises a facial interface 425 of a compliant material such as foam or rubber that contacts the face of the wearer, and that isolates an interior volume 430 of the body-worn device 405 from environmental light. The body-worn device 405 further comprises lenses 435-L, 435-R that are exposed to the interior volume 430 and are optically connected with one or more displays of the body-worn device 405. The body-worn device 405 further comprises circuitry (e.g., processor(s) and/or memory) that drives the displays to generate imagery that is transmitted through the lenses 435-L, 435-R (and in some cases, one or more intermediate optical components) to the eyes of the wearer. In some aspects, the circuitry (e.g., a network interface controller) is further operable to wirelessly connect with the optometric or ophthalmic device 105. For example, the optometric or ophthalmic device 105 may wirelessly transmit video or other signals during a procedure, which are received by the body-worn device 405 causing imagery to be displayed through the lenses 435-L, 435-R to the wearer, e.g., providing visualization of aspects of the procedure to the provider.

In some aspects, the body-worn device 405 further comprises an iris imager 440 representing one example of the iris imager 140. The iris imager 440 is exposed to the interior volume 430 and is communicatively connected with circuitry of the body-worn device 405. As shown, the iris imager 440 is disposed between the lenses 435-L, 435-R; however, other implementations may have the iris imager 440 in a different position, may have multiple iris imagers, and so forth. Thus, the iris imager 440 may be capable of unobtrusively acquiring images of the iris of the provider while the provider approaches or dons the body-worn device 405.

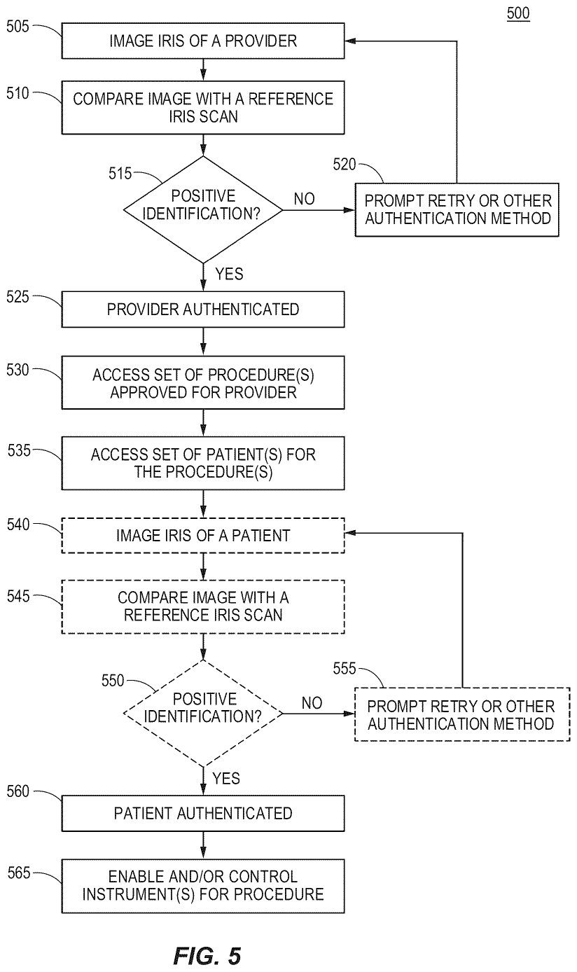

FIG. 5 is a method 500 of authenticating a provider and, optionally, a patient relative to a procedure using an optometric or ophthalmic device, according to one or more aspects. The method 500 may be used in conjunction with other aspects. For example, the method 500 may be performed using an authentication service, such as the authentication service 130 of FIG. 1.

The method 500 begins at block 505, where the authentication service acquires, using an iris imager that is communicatively connected with the optometric or ophthalmic device, an image of an iris of a provider. In some aspects, the iris imager (e.g., iris manager 140) is one of: integrated into the optometric or ophthalmic device; attached to the optometric or ophthalmic device; or integrated into a body-worn device and wirelessly connected to the optometric or ophthalmic device. In some aspects, acquiring the image of the iris of the provider may be responsive to the provider approaching the optometric or ophthalmic device (e.g., using a proximity sensor), or donning a body-worn device.

At block 510, the authentication service compares the image of the iris with a reference iris scan of the provider. In some aspects, comparing the reference the image of the iris with a reference iris scan of the provider comprises retrieving the reference iris scan from a network location (e.g., a remote electronic device, such as electronic device 150 in the example of FIG. 1).

In some aspects, the comparison of the image of the iris with a reference iris scan of the provider comprises determining, based on the image of the iris, whether the provider is wearing eye wear. Generally, eye wear such as glasses, contact lenses, safety glasses, face shields, etc. may affect the quality of the acquired image of the iris of the provider. In one non-limiting example, determining whether the provider is wearing eye wear comprises detecting and/or localizing the eye wear within the image. In another non-limiting example, determining whether the provider is wearing eye wear comprises determining whether a matching score (or other comparison metric) for the image of the iris and the reference iris scan is greater than a second threshold score that is less than a first threshold score corresponding to a positive identification of the provider. In some aspects, the second threshold score may be defined relative to the first threshold score (e.g., 85% of the first threshold score). In another non-limiting example, different approaches may be combined to determine whether the provider is wearing eye wear.

In some aspects, the comparison of the image of the iris with a reference iris scan of the provider further comprises adjusting one or more parameters of the comparison when the provider is wearing eye wear. For example, responsive to determining that the provider is wearing eye wear, the authentication service may apply a threshold score less than the first threshold score (whether the second threshold score or another value).

At block 515, the authentication service attempts to authenticate the provider using a comparison of the image of the iris with a reference iris scan of the provider. In some aspects, attempting to authenticate the provider comprises determining whether the matching score (or other comparison metric) is greater than the first threshold score corresponding to a positive identification of the provider. In some aspects, when the provider is wearing eye wear, attempting to authenticate the provider comprises determining whether the matching score (or other comparison metric) is greater than the adjusted threshold score.

If positive identification of the provider is not made (“No”), the authentication service determines that authenticating the provider using the comparison is not successful, and the method 500 proceeds to block 520, where the provider is prompted to retry the authentication or to perform another authentication method. In some aspects, prompting the provider is provided using a display or other output device communicatively connected with optometric or ophthalmic device.

If positive identification of the provider is made (“Yes”), the authentication service determines that authenticating the provider using the comparison is successful at block 525. At block 530, and responsive to authenticating the provider, the authentication service accesses a set of one or more procedures approved for the provider. In some aspects, accessing the set of one or more procedures comprises accessing a profile associated with the provider. At block 535, the authentication service accesses a set of one or more patients corresponding to the set of one or more procedures.

At an optional block 540, the authentication service acquires an image of an iris of a patient on whom the procedure is to be performed. The optional block 540 may be similar to block 505, whether performed using the same iris imager or another device. At an optional block 545, the authentication service compares the image of the iris with a reference iris scan of the patient. The optional block 540 may be similar to block 510.

At an optional block 550, the authentication service attempts to authenticate the patient using a comparison of the image of the iris of the patient with a reference iris scan of the patient. The optional block 550 may be similar to block 515. If positive identification of the patient is not made (“No”), the authentication service determines that authenticating the patient using the comparison is not successful, and the method 500 proceeds to an optional block 555, where the patient is prompted to retry the authentication or to perform another authentication method. If positive identification of the patient is made (“Yes”), the authentication service determines that authenticating the patient using the comparison is successful at the optional block 560.

At block 565, and responsive to determining that the procedure to be performed is included in the set of one or more procedures that are approved for the provider, the authentication service configures operation of the optometric or ophthalmic device to perform the procedure. In some aspects, configuring the operation of the optometric or ophthalmic device is further responsive to determining that the patient is included in the set of one or more patients.

In some aspects, configuring operation of the optometric or ophthalmic device to perform the procedure comprises applying one or more settings included in the profile associated with the provider. In some aspects, configuring operation of the optometric or ophthalmic device to perform the procedure comprises: transmitting a control signal that enables operation or controls one or more operational parameters of the optometric or ophthalmic device. The method 500 ends following completion of block 565.

As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network. An electronic device may be implemented with any suitable form factor, whether relatively static in nature (e.g., mainframe, computer terminal, server, kiosk, workstation) or mobile (e.g., laptop computer, tablet, handheld, smart phone, wearable device). The communicative capability between electronic devices may be achieved using any suitable techniques, such as conductive cabling, wireless transmission, optical transmission, and so forth.

Processors (e.g., processors 120, 155, etc.) are any electronic circuitry, including, but not limited to one or a combination of microprocessors, microcontrollers, application-specific integrated circuits (ASIC), application-specific instruction set processors (ASIP), and/or state machines, that is communicatively coupled to the memory and controls the operation of the system. In some aspects, the electronic circuitry is configured to perform any of the functions described herein. Further, the one or more processors are not limited to a single processing device and may encompass multiple processing devices.

The one or more processors may include other hardware that operates software to control and process information. In some aspects, the one or more processors execute software stored in the memory to perform any of the functions described herein. The one or more processors control the operation and administration of the electronic device by processing information (e.g., information received from input devices and/or communicatively coupled electronic devices).

The memory (e.g., memories 125, 160, etc.) may store, either permanently or temporarily, data, operational software, or other information for the one or more processors. The memory may include any one or a combination of volatile or non-volatile local or remote devices suitable for storing information. For example, the memory may include random-access memory (RAM), read-only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory, a disk, a CD, or a flash drive. In particular embodiments, the software may include an application executable by the one or more processors to perform one or more of the functions described herein.

### Example Embodiments

In certain embodiments, a system comprises an optometric or ophthalmic device, an iris imager, and one or more processors configured to acquire, using the iris imager, an image of an iris of a provider; attempt to authenticate the provider using a comparison of the image of the iris with a reference iris scan of the provider; responsive to authenticating the provider, access a set of one or more procedures approved for the provider; and responsive to determining that a procedure is included in the set of one or more procedures, configure operation of the optometric or ophthalmic device to perform the procedure.

In certain embodiments, a method of authenticating a provider prior to performing a procedure using an optometric or ophthalmic device comprises acquiring, using an iris imager that is communicatively connected with the optometric or ophthalmic device, an image of an iris of the provider; attempting to authenticate the provider using a comparison of the image of the iris with a reference iris scan of the provider; responsive to authenticating the provider, accessing a set of one or more procedures approved for the provider; and responsive to determining that the procedure is included in the set of one or more procedures, configuring operation of the optometric or ophthalmic device to perform the procedure.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
an optometric or ophthalmic device;
an iris imager; and
one or more processors configured to:
acquire, using the iris imager, an image of an iris of a provider;
attempt to authenticate the provider using a comparison of the image of the iris with a reference iris scan of the provider;
responsive to authenticating the provider, access a set of one or more procedures approved for the provider; and
responsive to determining that a procedure is included in the set of one or more procedures, configure operation of the optometric or ophthalmic device to perform the procedure.

2. The system of claim 1, wherein the iris imager is integrated into the optometric or ophthalmic device.

3. The system of claim 1, wherein the iris imager is attached to the optometric or ophthalmic device.

4. The system of claim 1, wherein the iris imager is integrated into a body-worn device and wirelessly connected to the optometric or ophthalmic device.

5. The system of claim 1, wherein:
the one or more processors are included in a first electronic device of the optometric or ophthalmic device.
the reference iris scan is stored in a second electronic device that is wirelessly connected with the first electronic device.

6. The system of claim 5, wherein the reference iris scan is associated with a profile of the provider that is stored in the second electronic device.

7. The system of claim 6, wherein configuring operation of the optometric or ophthalmic device to perform the procedure comprises applying one or more settings included in the profile.

8. The system of claim 5, wherein the one or more processors are further configured to:
access a set of one or more patients corresponding to the set of one or more procedures;
acquire an image of an iris of a patient on whom the procedure is to be performed; and
attempt to authenticate the patient using a comparison of the image of the iris of the patient with a reference iris scan of the patient that is stored in the second electronic device,
wherein configuring operation of the optometric or ophthalmic device is further responsive to determining that the patient is included in the set of one or more patients.

9. A method of authenticating a provider prior to performing a procedure using an optometric or ophthalmic device, the method comprising:
acquiring, using an iris imager that is communicatively connected with the optometric or ophthalmic device, an image of an iris of the provider;
attempting to authenticate the provider using a comparison of the image of the iris with a reference iris scan of the provider;
responsive to authenticating the provider, accessing a set of one or more procedures approved for the provider; and
responsive to determining that the procedure is included in the set of one or more procedures, configuring operation of the optometric or ophthalmic device to perform the procedure.

10. The method of claim 9, further comprising:
accessing a set of one or more patients corresponding to the set of one or more procedures;
acquiring an image of an iris of a patient on whom the procedure is to be performed; and
attempting to authenticate the patient using a comparison of the image of the iris of the patient with a reference iris scan of the patient,
wherein configuring the operation of the optometric or ophthalmic device is further responsive to determining that the patient is included in the set of one or more patients.

11. The method of claim 9, wherein the comparison of the image of the iris with a reference iris scan of the provider comprises:
determining, based on the image of the iris, whether the provider is wearing eye wear; and
adjusting one or more parameters of the comparison when the provider is wearing eye wear.

12. The method of claim 9, wherein configuring operation of the optometric or ophthalmic device to perform the procedure comprises:
transmitting a control signal that enables operation or controls one or more operational parameters of the optometric or ophthalmic device.

13. The method of claim 9, further comprising:
responsive to authenticating the provider, accessing a profile associated with the provider,
wherein configuring operation of the optometric or ophthalmic device to perform the procedure comprises applying one or more settings included in the profile.

14. The method of claim 9, further comprising:
determining that authenticating the provider using the comparison is not successful; and
prompting the provider to retry the authentication or to perform another authentication method.

15. The method of claim 9, wherein the iris imager is:
integrated into the optometric or ophthalmic device;
attached to the optometric or ophthalmic device; or
integrated into a body-worn device and wirelessly connected to the optometric or ophthalmic device.
